(19) United States
(12) Patent Application Publication
(10) Pub. No.: US 2003/0092885 A1
(43) Pub. Date: May 15, 2003

(54) Solubility and stability enhancement tag for structural and ligand binding studies of proteins

(75) Inventors: Pei Zhou, Durham, NC (US); Alexey Lugovskoy, Brighton, MA (US); Gerhard Wagner, Chestnut Hill, MA (US)

Correspondence Address: EDWARDS & ANGELL, LLP, P.O. BOX 9169, BOSTON, MA, 02209, US

(73) Assignee: President and Fellows of Harvard College

(21) Appl. No.: 10/210,186

(22) Filed: Jul. 31, 2002

Related U.S. Application Data
(60) Provisional application No. 60/309,290, filed on Jul. 31, 2001.

(51) Int. Cl. G01N 33/53; G06F 19/00; G01N 33/48; G01N 33/50; C07K 14/705

(52) U.S. Cl. 530/350; 435/7.1; 702/19

(57) ABSTRACT

The present invention provides methods of stabilizing proteins in solution. More particularly, the present invention provides a method of solublizing and stabilizing a target protein in solution by forming a fusion protein of the target protein with a small solubility and stability enhancing tag. The present invention also features methods of determining the structure of a target protein using a fusion protein to stabilize the target protein in solution.

### CROSS-REFERENCE TO RELATED APPLICATION

The present application is a continuation-in-part of U.S. Ser. No. 60/309,290 filed on Jul. 31, 2001; the disclosure of which application is incorporated herein by reference.

The present invention was supported by grants from the NIH, grant number GM 47467 and NSF, grant number MCB9316938. The U.S. Government may have certain rights to the present invention.

### BACKGROUND OF THE INVENTION

1. Field of the Invention.

The present invention provides methods of increasing solubility and stability in solution of a target protein. Moreover, the methods of the invention comprise providing a fusion protein comprising a solubility and stability enhancing tag and the target protein such that the solubility and stability of the fusion protein is greater than the stability and solubility of the unmodified target protein. The present invention further comprises methods of carrying out protein analytical techniques, such as spectroscopic methods, NMR, structural genomics, drug screening and proteomics wherein the increased solubility and stability of the fusion protein facilitates acquisition of analytical data.

2. Background

A serious problem limiting the study of proteins is the difficulty in preparing well-behaving protein samples. After an interesting protein is identified, typically it is necessary to overproduce the protein and to find conditions under which the expressed protein is stable and soluble at concentrations at least in the 100-μM range. Typically one or more analytical techniques are employed to characterize or study a protein of interest. Many analytical techniques require samples which are stable for the duration of the experiment. Making well behaved proteins efficiently is of particular interest. The major challenge in this approach is a robust preparation of the sample. For example, only 25% of overproduced proteins are biochemically stable and suitable for structural studies (Christendat, D., et al., (2000). Nat Struct Biol 7, 903-909).

Multiple approaches have been proposed to address this problem. Buffer conditions screening and introduction of point mutations in the protein of interest (Bagby, S., et al. (1997). J Biomol NMR 10, 279-82; Huang, B., et al. (1996). Nature 384, 638-641) have been useful in some systems. However, these methods are largely guided by trial and error, which makes them unsuitable for high throughput studies where extensive screening for large number of proteins is prohibitively costly.

Protein tags have been used extensively to enhance expression, stability and solubility of fusion proteins and facilitate their purification. For example, protein-fusion constructs have been used with great success for enhancing expression of soluble recombinant protein and as tags for affinity purification. Unfortunately, the most successful fusion tags, such as GST and MBP are large, which hinders direct NMR spectroscopy, crystallography studies and other analytical studies of the fusion proteins. Cleavage of the fusion proteins often re-introduces problems with solubility and stability.

Additionally, these large fusion tags, such as GST and MBP, have to be removed for structural studies. For X-ray structure determination, the high mobility of the protein tag, which is often independent of the protein of interest, interferes with crystallization and structure determination. Although independent mobility is of less of a concern in NMR spectroscopy, the size of most common protein tags, such as GST or MBP, is too large to make the structural characterization of a fusion protein by NMR possible.

Huth et al. provides NMR methods for determining the extent of protein folding for engineered protein domains of larger full-length proteins (Huth et al. (1997) Protein Science (5):2359-2364). Huth provides two T7 RNA polymerase-based expression vectors to express a fusion protein of the engineered protein with the B1 immunoglobulin binding domain of streptococcal protein G. Huth merely uses the fusion proteins to determine the level of folding of the engineered protein by NMR and then evaluates protein binding characteristics of the engineered protein based on the extent of folding.

The human genome project has led to the development of the field of proteomics which is the study of how proteins fold and interact to relate protein structure to protein function in order to identify and understand biological mechanisms. NMR gives information on the structure of proteins as they exist within biological complexes. This is a key advantage because no crystallization step is needed. The protein is scanned in solution, its natural environment, and therefore—unlike a crystallized molecule—is free to move as it would inside the cell of a living organism. Unfortunately, most proteins of interest are poorly soluble or unstable in solution.

Protein-fusion constructs have been used with great success for enhancing expression of soluble recombinant protein and as tags for affinity purification. Unfortunately, the most successful tags, such as GST and MBP are large, which hinders direct NMR studies of the fusion proteins. Cleavage of the fusion proteins often re-introduces problems with solubility and stability. It would be desirable to have methods of enhancing the stability and solubility of proteins with a fusion protein comprising the protein of interest and a small solubility and stability enhancing tag (small SSET) wherein the small SSET does not interact directly with the protein of interest. Further, it would be desirable to have methods of determining the structure by NMR techniques of a protein of interest using such fusion proteins. It would also be desirable to have methods of using such fusion proteins having a protein of interest and a small solubility and stability enhancing tag for proteomics, e.g., structural genomics studies.

### SUMMARY OF THE INVENTION

The present invention provides methods of enhancing protein solubility and stabilizing proteins in solution. Moreover, the present invention provides methods of performing analytical experiments such as spectroscopy, particularly NMR, structural genomics, drug screening and proteomics for target proteins which are poorly soluble or unstable in solution. Methods of the invention provide well-behaved protein samples wherein the protein samples are fusion proteins comprising a target protein of interest and a small solubility and stability enhancing tag.

The present invention provides methods of solubilizing and stabilizing a target protein in solution, the method comprising preparing a fusion protein, wherein the fusion protein comprises a small solubility and stability enhancing tag and the target protein such that the solubility and solution stability of the fusion protein are greater than the solubility and solution stability of the target protein.

The present invention also provides methods of collecting analytical data on a target protein, the method comprising the steps of

preparing a fusion protein, wherein the fusion protein comprises a small solubility and stability enhancing tag and the target protein;

performing an analytical technique using the fusion protein as a sample such that the fusion protein is substantially stable for the duration of the analytical technique.

Further, the present invention additionally provides methods of determining the structure of a target protein, the method comprising the steps of

providing a fusion protein, wherein the fusion protein comprises a small solubility and stability enhancing tag and the target protein such that the solubility and solution stability of the fusion protein are greater than the solubility and solution stability of the target protein;

collecting NMR spectroscopic data for the fusion protein; and

analyzing the collected NMR spectroscopic data for the fusion protein to determine the structure of the target protein.

Additionally, the small solubility and stability enhancing tags provided by the present invention can typically be used to prepare well-behaved fusion proteins for target proteins which are poorly soluble, unstable or susceptible to aggregation. Fusion proteins comprising a target protein and a small SSET are generally useful in structural genomics applications, proteomics and protein analysis experiments such as NMR experiments. Other applications of fusion proteins comprising a small SSET include screening drug leads such as substrates, inhibitors, agonists, antagonists and the like in NMR based structure-activity relationship investigations.

### BRIEF DESCRIPTION OF THE DRAWINGS

FIG. 1A is a ¹⁵N-HSQC NMR spectra of the free ¹⁵N-labeled DFF45 NTD (1-116);

FIG. 1B is a ¹⁵N-HSQC NMR spectra of the ¹⁵N-labeled DFF45 NTD (1-116) in complex with unlabeled DFF40 NTD (1-80). Arrows indicate distinct resonances of folded DFF45;

FIG. 1C is a ¹⁵N-HSQC spectra of the ¹⁵N-labeled chimeric gbDFF45 (12-100) in complex with unlabeled DFF40 NTD (1-80). Arrows indicate distinct resonances of folded DFF45;

FIG. 2 is a block diagram illustrating the interaction between DFF45 and DFF40 which effects correct folding of DFF40;

FIG. 3 is a block diagram illustrating the points of DFF45 where caspase-3 cleaves the protein thereby activating DFF40;

FIG. 4 is a series of two dimensional NMR spectrum of structured DFF40 NTD, unfolded DFF45 NTD, a protein that has low solubility (0.2 mM) and precipitates in about two days, and folded DFF45 NTD upon binding of DFF40 NTD to DFF45 NTD;

FIG. 5 is an illustration of the structure of DFF40/DFF45 CIDE domain complex;

FIG. 6 is an illustration of the structure of DFF40/DFF45 CIDE domain complex from another view; and

FIG. 7 is an illustration of the binding of the basic surface of DFF40 to the acidic surface of DFF45.

### DETAILED DESCRIPTION OF THE INVENTION

The present invention provides methods of increasing the stability and solubility of proteins. In general, the methods of the invention use fusion proteins that comprise a target protein and a small solubility and stability-enhancement tag (small SSET) such that the stability and solubility of the fusion protein is greater than the stability and solubility of the target protein. The methods of the invention are particularly suited for stabilizing proteins which are poorly soluble, are prone to precipitation or aggregation. The methods of increasing the stability and solubility of a protein are suitable for providing well-behaved protein samples for use in a variety of applications. While suitable applications for the methods of the invention are not particularly limited, preferred applications include stabilizing protein samples for spectroscopic analysis, structural studies by NMR, SAR by NMR, structural genomics, proteomics, drug screening methods, and high-throughput screening methods.

In one aspect of the invention, small solubility and stability enhancing tags can typically be used to prepare well-behaved protein samples for proteins which typically are not suitable for study with one or more protein analytical techniques. Preferred small SSET groups include B1 domains of protein G such as the 56 residue B1 domain of staphylococcal protein G, other B1 domains of other protein G, pancreatic trypsin inhibitor, and other small soluble protein sequences which are highly stable and highly soluble.

Analytical techniques suitable for use in the methods of the invention are not particularly limited. However, the methods of the invention are particularly suited for use in protein structural determinations using NMR, proteomics, drug screening, structural genomics, structure-activity-relationship studies, spectroscopy experiments and other analytical techniques common in protein characterization.

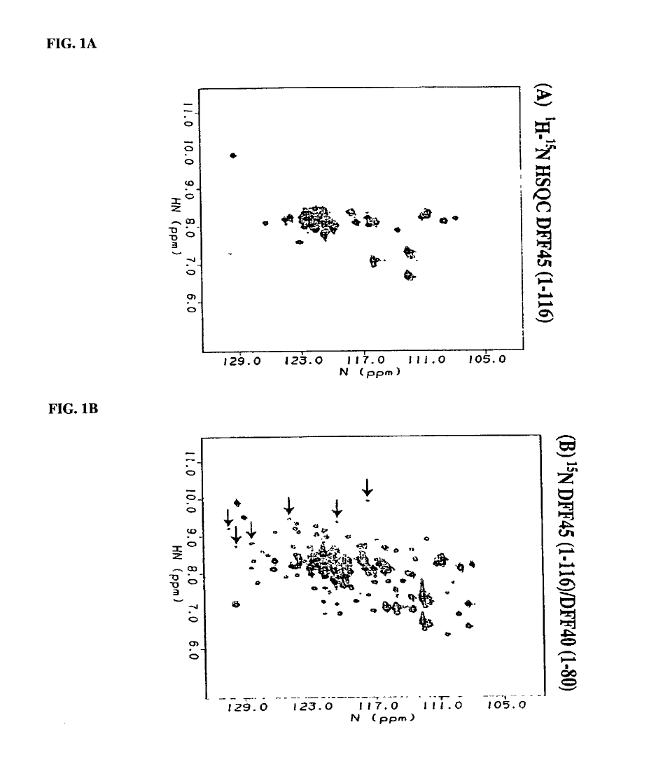

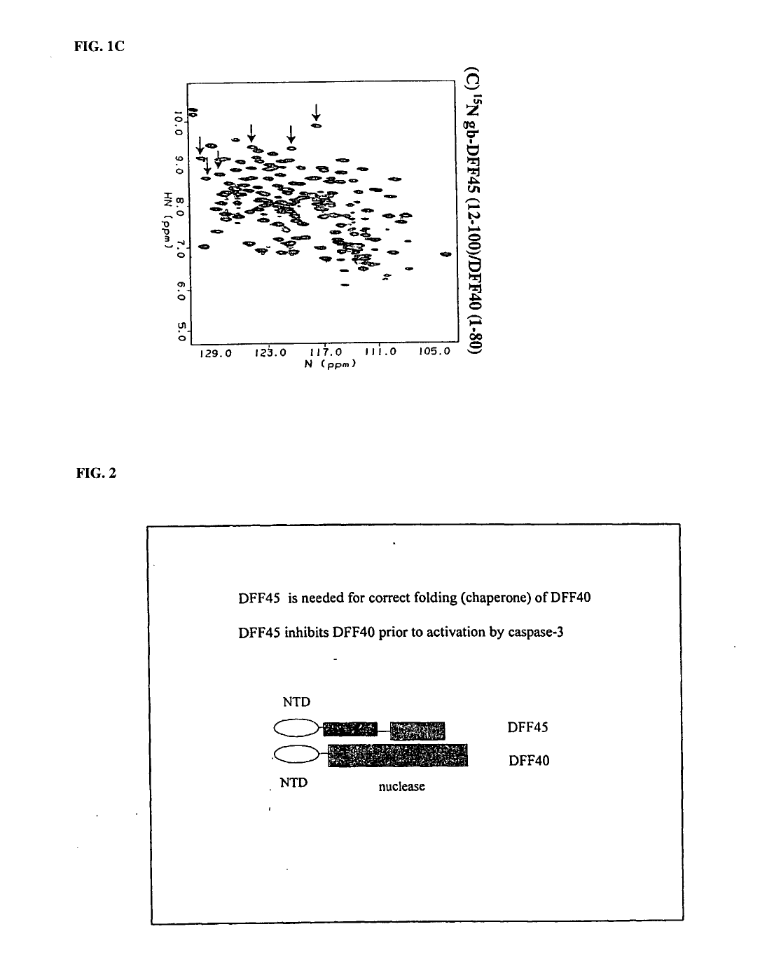

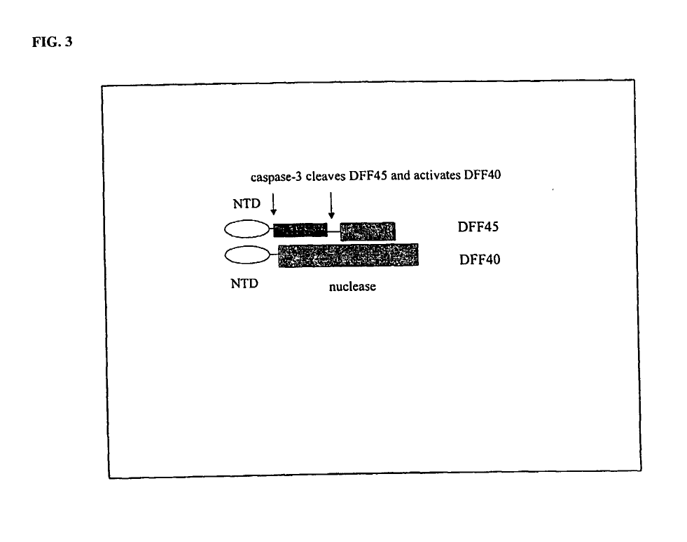

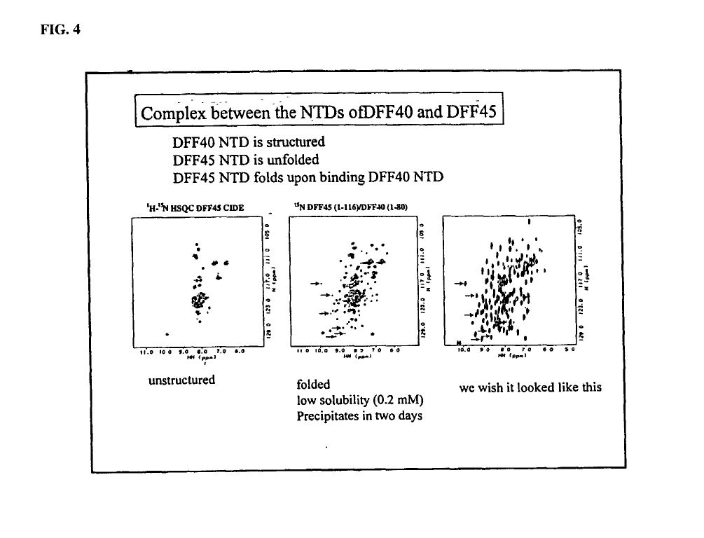

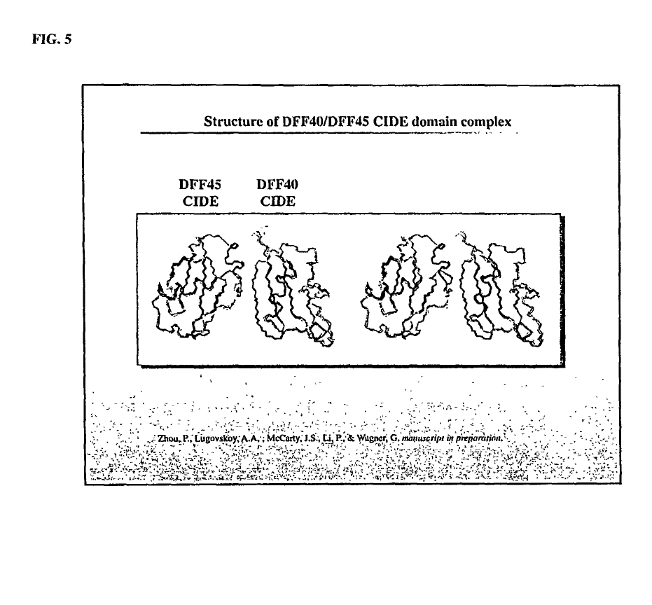

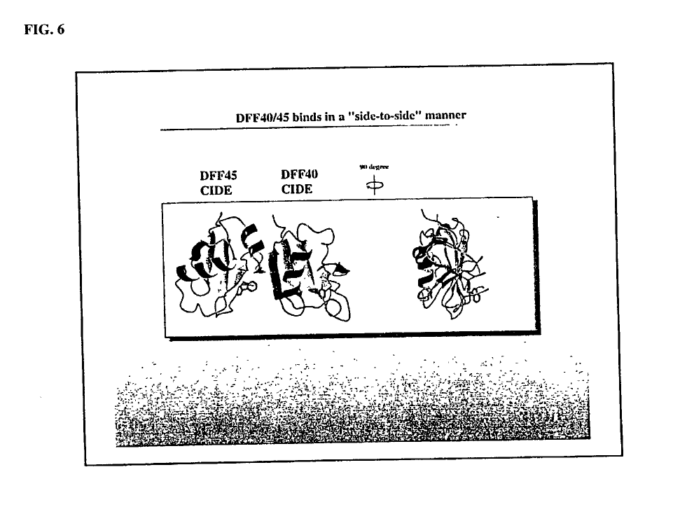

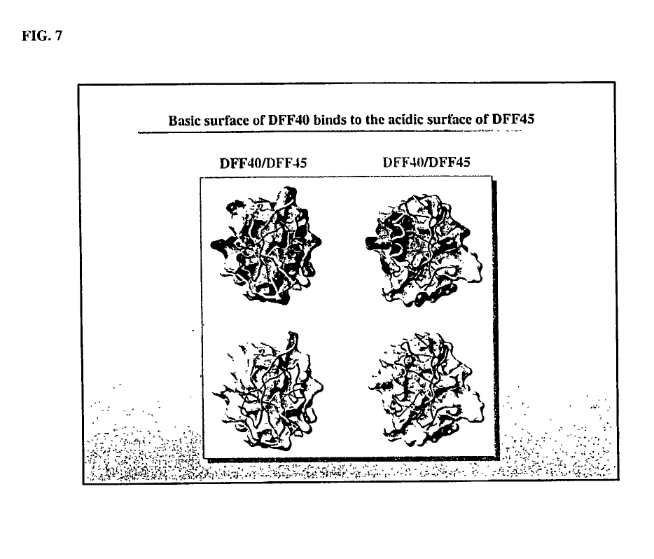

Typically, a small SSET does not substantively interact with the target protein such that the folding and protein interactions of a fusion protein comprising a small SSET are generally similar to the folding and protein interactions observed for the target protein in the absence of a small SSET. Typically, NMR spectra of the small SSET and a fusion protein comprising a target protein and the small SSET are collected. The contribution of the target protein to the NMR spectra of the fusion protein is generally determined by subtracting the signals for the NMR spectrum of the small SSET from the NMR spectrum of the fusion protein, see FIGS. 1A, 1B and 1C or FIG. 4 for a series of NMR spectra of a SSET, a wild type protein and a fusion protein comprising the SSET and the wild type protein.

In another aspect of the invention, methods are provided to determine the solution structure of a target protein by utilizing a fusion protein comprising the target protein and a suitable small SSET and collecting NMR data for the fusion protein. Spectral data for the target protein is typically obtained by subtracting the contributions of the small SSET from the spectral data of the fusion protein. Proteins which are typically unsuited for NMR spectroscopy due to insufficient protein solubility or instability are preferred proteins for use in the methods of structural determination by NMR provided by the invention.

Additionally, the NMR methods of the present invention are suitable for use in observing protein folding and protein interactions with other substances such as other proteins, DNA, drugs, substrates, ligands, receptors, inhibitors, and the like are elucidated by a variety of analytical techniques including NMR spectroscopy.

The methods of the present invention for collecting analytical data for proteins which are poorly soluble or unstable are also suitable for use in drug discovery applications. In a non-limiting example, the interactions of a potential drug candidate with a target protein can be assessed using structural studies by NMR. Alternatively, UV-Vis spectroscopy or another analytical technique can be used to measure binding affinity of a variety of drug candidates with a target protein. In general, the methods of the present invention are suitable for any method of drug screening or discovery in which increasing the stability and solubility of a protein target is beneficial.

The present invention provides methods of solubilizing and stabilizing a target protein in solution, the method comprising preparing a fusion protein, wherein the fusion protein comprises a small solubility and stability enhancing tag and the target protein such that the solubility and solution stability of the fusion protein are greater than the solubility and solution stability of the target protein.

Preferred methods of solubilizing and stabilizing a target protein include the use of fusion proteins having a linker sequence disposed between the target protein and the small solubility and stability enhancing tag. Particularly preferred are fusion proteins wherein the small solubility and stability enhancing tag does not substantially interact with the target protein.

Other preferred methods of solubilizing and stabilizing comprise a fusion protein having a solubility of at least about 50 μM, more preferably a solubility of at least about 100 μM. Particularly preferred methods of solubilizing and stabilizing proteins comprise a fusion protein having a solubility of at least about 250 μM. Preferably the solubility of the fusion protein is at least about twice the solubility of the target protein. More preferably the solubility of the fusion protein is at least about five times or about ten times the solubility of the target protein.

Additional preferred methods of solubilizing and stabilizing comprise a fusion protein which is stable for at least about seven days, more preferably the fusion protein is stable for at least about 30 days. In general, the invention provides methods which increase the stability of a target protein by a factor of two, more preferably increase the stability of the target protein by a factor of about five. Particularly preferred methods of the invention increase the stability of a target protein by a factor of about 10.

The present invention also provides methods of collecting analytical data on a target protein, the method comprising the steps of

preparing a fusion protein, wherein the fusion protein comprises a small solubility and stability enhancing tag and the target protein;

performing an analytical technique using the fusion protein as a sample such that the fusion protein is substantially stable for the duration of the analytical technique.

Analytical techniques suitable for use in the methods of the present invention are not particularly limited and include techniques commonly used to characterize proteins. Preferred techniques include spectroscopic techniques such as NMR, ESR, UV, UV-Vis, Raman, IR and the like. Particularly preferred are NMR techniques, especially multi-dimensional, solution-phase NMR.

The present invention also provides methods of determining the structure of a target protein, the method comprising the steps of

providing a fusion protein, wherein the fusion protein comprises a small solubility and stability enhancing tag and the target protein such that the solubility and solution stability of the fusion protein are greater than the solubility and solution stability of the target protein;

collecting NMR spectroscopic data for the fusion protein; and

analyzing the collected NMR spectroscopic data for the fusion protein to determine the structure of the target protein.

The present invention provides a small protein-fusion construct that does not interfere with direct NMR studies of the fusion protein.

Proteins suitable for use as target proteins in the methods of the present invention are not particularly limited. In general, proteins which exhibit poor solubility, have low stability or are prone to aggregation are preferred. Preferred target proteins for use in methods of collecting analytical data are only limited by the requirements of a specified analytical technique. For example, protein structural determination using NMR spectroscopy are generally limited to proteins weighing less than about 50 kD, preferably less than about 40 kD. Particularly preferred are proteins weighing less than about 30 kD for use in methods of protein structural determination by NMR.

Small solubility and stability enhancing tags (small SSET) suitable for use of the present invention include any small, soluble and highly stable molecule. Preferred small SSET's suitable for use in the present invention are proteins which have less than about 200 amino acid residues, preferably less than about 175, 150 or 125 amino acid residues. Particularly preferred small SSET proteins suitable for use in the present invention have between about 30 and 100 amino acid residues, or more preferably between about 40 and 80 amino acid residues or between about 50 and 70 amino acid residues. Particularly preferred examples of small solubility and stability enhancing tags for use in fusion proteins and methods of the invention include the B1 domain of staphylococcal protein G having 56 residues, 58 residue basic pancreatic trypsin inhibitor (BPTI), SH3, SH2, CARD domain, and any other naturally occurring small, highly soluble and stable proteins and engineered highly soluble proteins having between about 30 and 90 residues.

As used herein solution and solution stability generally refer to an aqueous media and to the stability of a substance in said aqueous media. The aqueous media includes pure water and water solutions comprising salts, buffers, inorganic compounds and salts, organic compounds and salts, proteins, DNA, polymers or any combination thereof.

Small solubility and stability enhancing tags of the invention may be coupled to a target protein directly or through a linker. The mode of coupling, while not particularly limited, includes covalent bonds such as amine, ester, disulfide and other covalent bonds, hydrogen bonding arrays, electrostatic interactions and the like. The small SSET may be attached at any point of the target protein including the N-terminus, the C-terminus and sidechain functional groups of one or more amino acid residues. Preferably the small SSET is coupled to the target protein at the N-terminus or the C-terminus. Particularly preferred are fusion proteins wherein the small SSET is covalently bonded to the N-terminus of the target protein.

Other small, stable and highly soluble SSETs, such as the 58-residue basic pancreatic trypsin inhibitor (BPTI), which is soluble up to 60 mM (G. Wagner, Ph.D. thesis, Konformation und Dynamik von Protease-Inhibitoren: ¹HNMR-studien, Eidenoessischen Technischen Hochschule, Zurich (1977)), and other small and highly soluble proteins are suitable for use in the methods of the present invention. Further engineered protein oligiomers that are optimized for solubility are also suitable for use in the methods of the invention.

In certain preferred embodiments, a linker may be disposed between the small SSET and the target protein. Preferred linkers comprise about 1 to about 20 amino acid residues. Particularly preferred linkers comprise about 3 to 15 amino acid residues or about 5 to 10 amino acid residues. Preferred linkers for use in fusion proteins of the invention are non-cleavable, e.g., non-cleavable linkers to not comprise a residue or sequence of residues that is targeted by one or more proteases or other selective protein sequence cleaving agents. However, in certain preferred embodiments, cleavable linker groups may be suitable for use in the methods of the invention.

As used herein and in the claims, the phrase “an amino acid side chain” refers to the distinguishing substituent attached to the α-carbon of an amino acid; such distinguishing groups are well known to those skilled in the art. For instance, for the amino acid glycine, the side chain is H; for the amino acid alanine, the side chain is CH₃, and so on.

As used herein and in the claims, the term “amino acid” is intended to include common natural or synthetic amino acids and common derivatives thereof, known to those skilled in the art. Typical amino-acid symbols denote the L configuration unless otherwise indicated by a D appearing before the symbol.

Particularly preferred small SSET groups suitable for use in the methods of the invention include the 56 amino acid protein G B1 domain which is highly stable and soluble molecule. The complete assignment of the chemical shifts for the NMR spectra for the protein G B1 domain has been reported (Gronenborn, et al. (1991). Science 253, 657-61). The application provides methods using a non-cleavable protein G B1 tag to solubilize and stabilize the NMR samples during the process of structure determination. In an exemplary example of the methods of the invention, a non-cleavable protein G B1 domain was used as a small SSET to stabilize and solubilize target proteins thereby facilitating NMR data collection and protein structure determination of the heterodimeric complex between regulatory domains of human DNA fragmentation factor 40 (DFF40) and human DNA fragmentation factor 45 (DFF45) CIDE domains.

The methods of the invention using small SSET provide a robust and straightforward way to produce biochemically well-behaving NMR samples which are stable for extended periods of time and are highly soluble for proteins that are insufficiently soluble and stable by themselves.

The present invention also provides methods of stabilizing and solubilizing proteins for structural genomics studies. The methods of structural determination by NMR of the present invention permit rapid determination by NMR of the present invention permit rapid determination of how proteins fold. In the Examples, the folding of a chimeric protein was investigated by selecting highly charged, soluble, but yet small protein G B1 domain as a tag to solubilize the proteins of the chimeric protein. In general, any protein or protein domain with a molecular weight of less than about 40 kDa, or preferably less than about 30 kDa, would be suitable for the structural genomics studies using methods of the present invention.

The present invention provides methods of using small solubility and stability enhancing tags such as protein G B1 domain, to stabilize and solubilize target proteins. The methods of stabilizing and solubilizing proteins are appropriate for a variety of applications which are not particularly limited. Preferred applications include drug screening, structural determination by NMR, structural genomics and proteomics. The methods of the invention result in a significant improvement in solubility and stability of the sample, which enabled the detailed structural characterization of this complex system. See for example Zhou et al., (2001) J. Biomolecular NMR, 20, 11-14.

In a non-limiting example, the methods of the present invention are used to solublize and stabilize the complex between the regulatory domains of the DNA Fragmentation Factor 40 (DFF40) and DFF45. As described in Example 1, a highly soluble B1 domain of the staphylococcal protein G (56 residues) has been attached to the N-terminus of the DFF45 domain such that the fusion protein comprising DFF45 and B1 domain of protein G and the complex of the fusion protein with DFF40 are more soluble and more stable than the complex with out a small SSET present. The wild-type protein complex precipitates in less than 2 days, the SSET-complex is stable for >30 days. The B1 domain of protein G is small enough so that the complex structure could be solved by NMR techniques with the SSET attached. No interactions between the SSET and the protein of interest were observed indicating that the SSET does not affect structure and function of the protein of interest. The methods of the invention are generally useful for structural studies of poorly behaving proteins and for NMR-based screening for drug leads.

### EXAMPLE 1

### Preparation of a Chimeric Protein Containing Protein G B1 Domain and DFF45 CIDE Domain

The chimeric protein containing protein G B1 domain and DFF45 CIDE domain was generated by 2-step PCR using three primers (1) 5′-GGA GAT ATA CAT ATG CAG TAC AAG CTT ATC CTG-3′; (2) 5′-TAG AGT CCG GAT CTC GCC AGA TTC GGT TAC CGT GAA GGT TTT-3′; (3) 5′-GCA GCC GGA TCC TCA ATC TGA ATC TGA ATT GTT GTA TGC CCA 3-′. First PCR reaction was carried out using primer 1 and primer 2 encoding residues 1-56 of the protein G B1 domain and the first six residues of DFF45 CIDE domain (S12-L17). A second PCR reaction was carried out using the PCR product from the previous reaction and the third primer was used to obtain the final DNA insert containing a chimeric protein (protein G B1 M1-E56 and DFF45-CIDE S12-D100), which we call gbDFF45-CIDE in the later discussions. This insert was cloned in a pET30 a(+) vector between the Nde I site and BamHI site and the fusion protein was overproduced in Escherichia coli BL21(DE3) cell line.

### EXAMPLE 2

### Over-Expression and Purification of gbDFF45 CIDE (12-100), DFF45 CIDE (12-100) and their Complex with DFF40 CIDE (1-80)

The DFF45 CIDE domain (12-100) was cloned into pGEX6P2 vector. The CIDE domain of DFF40 1-80 was sub-cloned into pET30 a(+) with the His₆-tag fused at the C-terminus. Cells transformed with GST-fused DFF45 CIDE or gbDFF45 CIDE were grown at 37° C. and induced with 1 mM isopropyl-D-thiogalactoside at 20° C. in M9-minimal media supplemented with ¹⁵N-NH4Cl (1 g/L) for production of ¹⁵N labeled protein. Unlabeled DFF40 CIDE was obtained in a similar way except for growing cells in LB-media. The cell pellets of ¹⁵N-labeled GST-DFF45 CIDE and ¹⁵N-gbDFF45 CIDE were mixed with unlabeled DFF40 CIDE prior to sonication. The complexes between GST-DFF45/DFF40 CIDE complex and gbDFF45/DFF40 CIDE complex are purified by Ni²⁺ NTA affinity chromotography using manufacturer's protocol (Qiagen). GST was removed by cleavage with Prescission protease (Amersham Pharmacia) at 4° C. for 2 hours. Purified DFF40/45 CIDE complexes were exchanged into NMR buffer containing 20 mM phosphate, 50 mM NaCl, 5 mM DTT in H₂O/D₂O(9/1).

### EXAMPLE 3

### A Protein G Tagged CIDE/CIDE Complex has a Superior Biochemical Behaviour and Displays Higher Quality NMR Spectra

The quality of the ¹H-¹⁵N heteronuclear single quantum coherence (HSQC) spectrum is a sensitive measure of the biochemical behaviour of the protein in solution. We used such spectra to examine the solution behavior of the N-terminal domain of DFF45. FIG. 1A shows that this domain has very little dispersion of its cross peaks indicating that it is primarily unfolded. When adding unlabeled N-terminal domain of DFF40 (1-80) the dispersion of the HSQC spectrum increases dramatically (FIG. 1B) indicating that the protein folds upon binding DFF40. However, the complex has very low solubility and precipitates within days. This changed dramatically when we used the SET approach. When ESN gbDFF45 was used to form the complex with DFF40 the quality of the HSQC spectrum increased dramatically (FIG. 1C). In order to quantitatively compare the properties of the complex an HSQC spectrum of the untagged complex was compared with that of a complex where DFF45 was fused with the SET, recorded under exactly the same experimental conditions. The superior quality of the SET-complex is obvious. Furthermore, addition of the SET increased the solubility of the complex three fold (from 0.2 mM to 0.6 mM). The stability of the sample increased approximately 6 fold (from 5 days to >30 days at 23° C.).

A substantial problem with the use of a fusion protein for NMR studies, is an increase in spectra complexity. However, in our case the attachment of protein G B1 tag caused only little spectral overlap. In addition, the resonance frequences of protein G B1 tag are very similar to these of free protein G B1 domain, and thus can be quickly identified.

### EXAMPLE 4

### The Protein G B1 Tag Does Not Interact with the CIDE/CIDE Complex

A common concern about the use fusion proteins for structural studies is that the protein tag may interfere the physical properties of the protein of interest. This seems to be a particular problem when the protein tag is bigger than a protein of interest. To examine this possibility we carefully examined the ¹⁵N and ¹³C NOESY spectra of gbCIDE/CIDE complex. Despite a careful examination, we did not observe any interdomain NOEs between the CIDE/CIDE complex and the attached protein G B1 tag, indicating that the latter is not packing against either of the CIDE domains. This observation is further supported by a distinct relaxation behavior and narrow line-widths of the protein G B1 tag resonances, compared to those of the CIDE/CIDE complex (date not shown). To analyze the spectra of the complex we used TROSY-type spectra (Pervushin et al., 1997; Salzmann et al., 1999) and found them to be especially beneficial for this situation. The intensities of the resonances of the slowly tumbling CIDE/CIDE complex were significantly enhanced compared to those of rapidly tumbling protein G B1 tag. We attribute the distinct NMR properties of protein G B1 tag to its high acidic/basic nature, which causes it to be solvent accessible, rather then the packing against CIDE/CIDE complex.

### EXAMPLE 5

### A Protein G Tagged Construct of eIF4E (Mouse) has Superior Solubility and Stability

The eukatyotic translation initiation factor eIF4E (mouse) is sparingly soluble, e.g., only soluble in aqueous media up to about 0.1 mM, and precipitates from solution within about a week: A N-terminal fusion protein of eIF4E and Protein G tag was prepared by the procedures of Examples 1 and 2 which results in a fusion protein with a solubility of up to 0.6 mM, and the fusion protein does not precipitate from solution for at least a month. The preparation of the fusion protein comprising eIF4E and Protein G tag made NMR assignments of the eIF4E translation initiation factor possible.

### EXAMPLE 6

### The Protein G Tag Increases the Stability of the Protein FAIM

The FAIM protein, a 20 kDa apoptosis inhibitor in b cells can be concentrated up to 1 mM. However, at 25° C., the untagged protein irreversibly precipitates from solution within about one week. A fusion protein of the FAIM protein and the Protein G tag was prepared by the procedures of Examples 1 and 2. The fusion protein is stable for at least one month in solution with no precipitation occurring during that time period.

Although a preferred embodiment of the invention has been described using specific terms, such description is for illustrative purposes only, and it is to be understood that changes and variations may be made without departing from the spirit or scope of the following claims. All references cited herein are incorporated by reference into the present application.

What is claimed is:

1. A method of solublizing and stabilizing a target protein in solution, the method comprising preparing a fusion protein, wherein the fusion protein comprises a small solubility and stability enhancing tag and the target protein such that the solubility and solution stability of the fusion protein are greater than the solubility and solution stability of the target protein.

2. A method of claim 1, wherein the fusion protein comprises a linker sequence disposed between the target protein and the small solubility and stability enhancing tag.

3. A method of claim 1 or claim 2, wherein the small solubility and stability enhancing tag and the target protein do not substantially interact.

4. A method of claim 1, 2 or 3, wherein the small solubility and stability enhancing tag is a peptide with a solubility of at least about 5 mM which comprises less than about 200 amino acid residues.

5. A method of claim 4, wherein the small solubility and stability enhancing tag comprises between about 30 and 200 amino acid residues

6. A method of any one of claims 1-5, wherein the small solubility and stability enhancing tag is a protein selected from the group consisting of protein G B1 domains, BPTI, SH3, SH2, CARD domain and other highly soluble protein domains.

7. A method of any one of claims 1-6, wherein the solubility of the fusion protein is at least about twice the solubility of the target protein.

8. A method of claim 7, wherein the solubility of the fusion protein is at least about five times greater than the solubility of the target protein.

9. A method of any one of claims 1-8, wherein the stability of the fusion protein in solution is at least twice the stability of the target protein in solution.

10. A method of claim 9, wherein the stability of the fusion protein in solution is at least five times that of the stability of the target protein in solution.

11. A method of claim 9, wherein the stability of the fusion protein in solution is at least an order of magnitude greater than the stability of the target protein in solution.

12. A method of claim 9, wherein the stability of the fusion protein in solution is at least about 7 days.

13. A method of claim 12, wherein the stability of the fusion protein in solution is at least about 30 days.

14. A method of collecting analytical data on a target protein, the method comprising the steps of
preparing a fusion protein, wherein the fusion protein comprises a small solubility and stability enhancing tag and the target protein;
performing an analytical technique using the fusion protein as a sample such that the fusion protein is substantially stable for the duration of the analytical technique.

15. A method of claim 14, wherein the analytical technique is chosen from the group consisting of NMR, SAR by NMR, ESR, UV, UV-Vis, Raman, IR, mass spectroscopy, binding assays, drug screening methods and high throughput screening techniques.

16. A method of claim 14, wherein the analytical technique is solution phase NMR.

17. A method of claim 16, wherein the fusion protein is substantially stable in a NMR solvent for at least the duration of one protein NMR spectroscopy experiment.

18. A method of claim 16, wherein the fusion protein is substantially stable in a NMR solvent for at least about seven days.

19. A method of clam 16, wherein the fusion protein is substantially stable in a NMR solvent for at least about thirty days

20. A method of any one of claims 14-19, wherein the fusion protein comprises a linker sequence disposed between the target protein and the small solubility and stability enhancing tag.

21. A method of any one of claims 14-20, wherein the small solubility and stability enhancing tag and the target protein do not substantially interact.

22. A method any one of claims 14-21, wherein the small solubility and stability enhancing tag is a peptide having a solubility of at least about 5 mM and comprising less than about 200 amino acid residues.

23. A method of claim 22, wherein the small solubility and stability enhancing tag comprises between about 30 and 100 amino acid residues

24. A method of any one of claims 14-23, wherein the small solubility and stability enhancing tag is a protein selected from the group consisting of protein G B1 domains, BPTI, SH3, SH2, CARD domain and other highly soluble protein domains.

25. A method of any one of claims 14-24, wherein the fusion protein comprises a linker peptide sequence disposed between the target protein and the small solubility and stability enhancing tag.

26. A method of any one of claims 14-25, wherein the solubility of the fusion protein is at least about twice the solubility of the target protein.

27. A method of claim 26, wherein the solubility of the fusion protein is at least about five times greater than the solubility of the target protein.

28. A method of any one of claims 14-27, wherein the stability of the fusion protein in solution is at least twice the stability of the target protein in solution.

29. A method of claim 28, wherein the stability of the fusion protein in solution is at least five times that of the stability of the target protein in solution.

30. A method of claim 28, wherein the stability of the fusion protein in solution is at least an order of magnitude greater than the stability of the target protein in solution.

31. A method of determining the structure of a target protein, the method comprising the steps of
providing a fusion protein, wherein the fusion protein comprises a small solubility and stability enhancing tag and the target protein such that the solubility and solution stability of the fusion protein are greater than the solubility and solution stability of the target protein;
collecting NMR spectroscopic data for the fusion protein; and
analyzing the collected NMR spectroscopic data for the fusion protein to determine the structure of the target protein.
